(19) United States
(12) Patent Application Publication
(10) Pub. No.: US 2015/0238746 A1
(43) Pub. Date: Aug. 27, 2015

(54) FRANGIBLE CONNECTOR CLOSURE FOR TUBULAR CONNECTOR

(71) Applicants: BAXTER HEALTHCARE SA, Glattpark (Opfikon) (CH); BAXTER INTERNATIONAL INC., Deerfield, IL (US)

(72) Inventors: Eugen Nisipeanu, Wadsworth, IL (US); Atif Mehmet Yardimci, Lake Forest, IL (US); Christophe Andre Joseph Montavon, Ixelles (BE); John William Putnam, Lindenhurst, IL (US); Gilbert Albert Semaan, Basel (CH); Arnaud Jaspard, Havre (BE); Haitao Wang, Shanghai (CN); David Dawei Hsu, Evanston, IL (US)

(73) Assignees: BAXTER HEALTHCARE SA, Glattpark (Opfikon) (CH); BAXTER INTERNATIONAL INC., Deerfield, IL (US)

(21) Appl. No.: 14/190,395

(22) Filed: Feb. 26, 2014

(51) Int. Cl. A61M 39/10; A61M 39/20

(52) U.S. Cl. CPC A61M 39/10; A61M 39/20; A61M 2039/1061

(57) ABSTRACT

A frangible connector closure is provided for connection to a medication administration tube, and includes a tubular body portion having an open end configured for being connected to a supply of medication, and an opposite closed end. An elongate, rigid frangible member is disposed at the closed end and closes the end from fluid flow. The frangible member has a closure end and an opposite free end, and a longitudinal axis. In addition, the frangible member extends in an axial direction and has a member transverse dimension. The free end forms a finger portion extending in the axial direction and has a transverse dimension less than the member transverse dimension. The closure end of the frangible member is readily separable from the body portion by manual manipulation relative to the body portion when the frangible member is disposed inside the tube, for user-activated opening of the connector.

### BACKGROUND

The present invention relates generally to closures used with medication administration sets, and more specifically relates to such closures where the closure is opened by external manipulation of the closure while it is inserted in a flexible tube, such that upon opening of the closure, a frangible portion breaks free and is carried by fluid flow within the tube.

Medications, which in this application refer to any flowable medicinal substance, including but not limited to medicine, blood, plasma, saline solutions, or the like, are often provide to the patient in flexible plastic bags. Control of the administration of the medication to the patient is achieved through an assembly of components known as an administration set. Administration sets include, among other things at least one flexible bag or container of medicine, at least one length of flexible tubing, connectors attached to ends of the tubing, and control valves or clamps. In some cases, the administration set is provided in a sterile pouch or bag, including all of the components. Such administration sets are used both by medical technicians in hospitals or clinics, and also by patients who self-administer medication. One example of patient-controlled medicinal administration is peritoneal dialysis.

Frangible connectors are known in the art for controlling medication flow in such administration sets. A suitable connector is disclosed in U.S. Pat. No. 4,181,140. The frangible connector is located between the flexible container of medicine and a length of tubing. A frangible end of the connector is inserted into the tubing, and while the end is connected to the connector, fluid flow from the container is blocked. When the user or technician subjects the frangible end to manipulation, such as in a twisting or bending action, the frangible end breaks free from the connector, initiating fluid flow from the flexible container, through the tube for administration to the patient. The separated frangible end flows within the tube to the next component or obstruction.

Conventional frangible connectors are designed so that once separated, the frangible end does not unduly restrict medicinal flow within the tube towards the patient. However, in some cases, medicinal flow rate in the tube has been less than desired. Also, conventional separated frangible ends have in some situations become caught on the inside surface of the tube after separation. Thus, there is a need for an improved frangible connector that is designed to address these design drawbacks of conventional structures.

### SUMMARY

The above-identified need is met by the present frangible connector closure for a flexible tube, wherein a tubular body has a frangible member disposed at one end for preventing fluid flow through the connector prior to separation. As is the convention in the administration art, the frangible member is inserted into an end of a flexible tube, and the opposite end of the tube is ultimately connected to a fitting used for administering a flowable medication to a patient.

A finger is provided at a free end of the frangible piece, and axially extending, laterally projecting standoffs are dimensioned so that the frangible connector flows within the tube freely and only abuts the next component or tubing constriction, such as created by a clamp, without obstructing the fluid path. The frangible connector closure is the closure of the tubular connector, connected to the medication bag port. To activate the transfer of the solution from a medication bag, the medical technician or the patient bends and breaks the part by separating the frangible portion from the tubular body. The broken frangible member slips down inside the tubing up to the next component or constriction in the line, as caused by a shutoff valve. The single finger tip avoids obstruction of the fluid path.

More specifically, a frangible connector for a tubular connector is provided for connection to a medication administration tube, and includes a tubular body portion having an open end configured for being connected to a supply of medication, and an opposite closed end. An elongate, rigid frangible member is disposed at the closed end and closes the end from fluid flow. The frangible member has a closure end and an opposite free end, and a longitudinal axis. In addition, the frangible member extends in an axial direction and has a member transverse dimension. The free end forms a finger portion extending in the axial direction and having a transverse dimension less than the member transverse dimension. In addition, the closure end of the frangible member is constructed to permit ready separation from the body portion by manual manipulation of the frangible member relative to the body portion when the frangible member is disposed inside the tube, for user-activated opening of the connector.

### BRIEF DESCRIPTION OF THE DRAWINGS

FIG. 1 is a fragmentary elevation of an administration set equipped with the present frangible connector closure;

FIG. 2 is a front perspective view of the present frangible connector closure;

FIG. 3 is a side elevation of the connector of FIG. 2;

FIG. 4 is a cross-section taken along the line 4-4 of FIG. 3 in the direction generally indicated; and

FIG. 5 is a side elevation of the present connector in a flexile tube with the frangible member shown separated and lodged in the tube.

### DETAILED DESCRIPTION

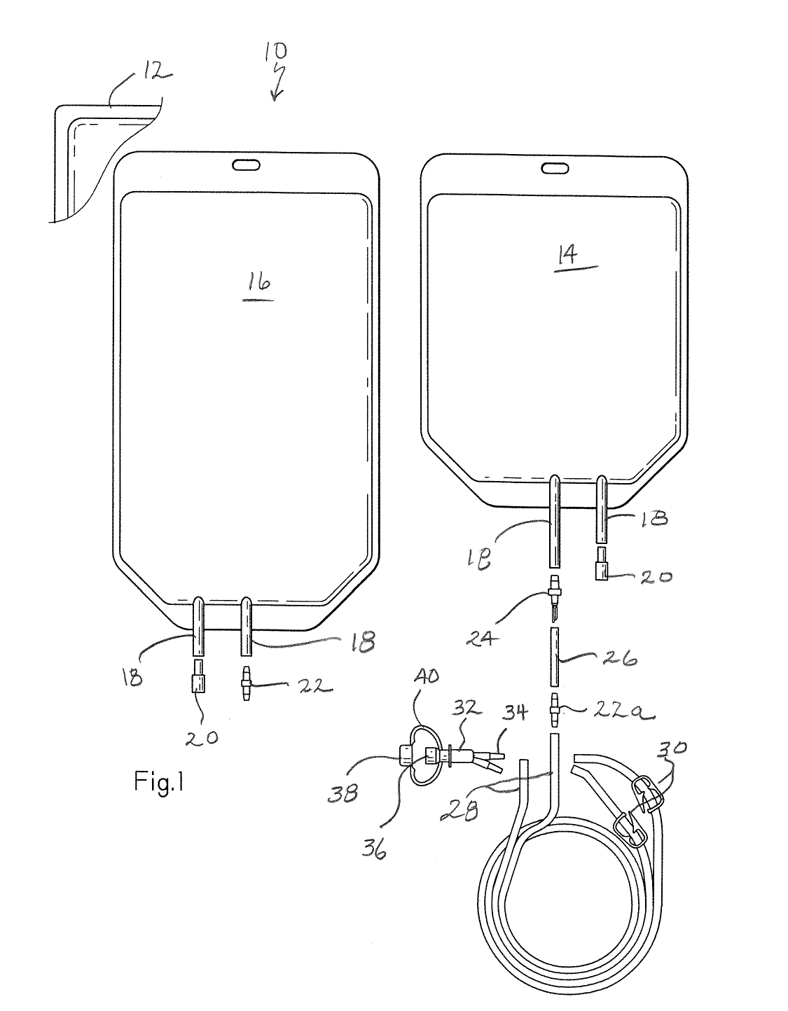

Referring now to FIG. 1, a medicinal administration set is generally designated 10, and is of the type commonly used in hospitals, clinics and for home use for administration of medications. An overpouch 12, preferably of flexible plastic material or the like encloses the components and maintains sterility. A medication bag 14 is also flexible, and is optionally accompanied by a supplemental medication bag 16, provided with a secondary medication. Both bags 14, 16 are provided with at least one and preferably two tubular ports 18. One port 18 on each bag 14, 16 is provided with a dual chamber bag cap 20, and the other is provided with a dual shaft port tube/tube coupler 22, or the present frangible connector closure 24, described in greater detail below.

A length of flexible tubing 26 is placed over the frangible connector closure 24, and through a second port tube/tube coupler 22a, is placed in fluid communication with a length of flexible administration tubing 28. In the preferred embodiment two lengths of tubing 28 are provided, each with a suitable releasable line clamp 30 for controlling flow in the respective tube. Also, a Y-junction fitting 32 is provided that is connectable via inlet ports 34 to each of the two lengths of tubing 28, and has an outlet 36 with a cap 38. In the preferred embodiment, the cap 38 is provided with a pull ring 40, acting as a sterility protector, as is known in the art.

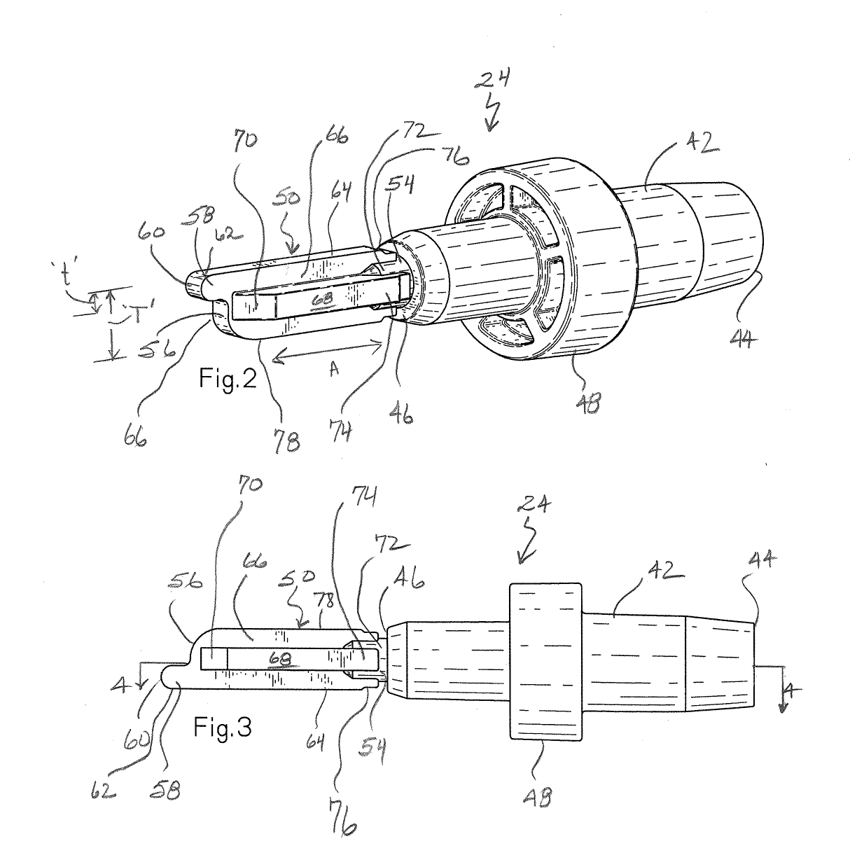

Referring now to FIGS. 2-4, the present frangible connector coupler 24 is shown in greater detail. A tubular body portion 42 has an open end 44 configured for being connected to a supply of medication through the bag port 18, and an opposite closed end 46. Between the ends 44, 46 is a radially projecting ring 48 serving as an installation gripping aid and a tubing stop.

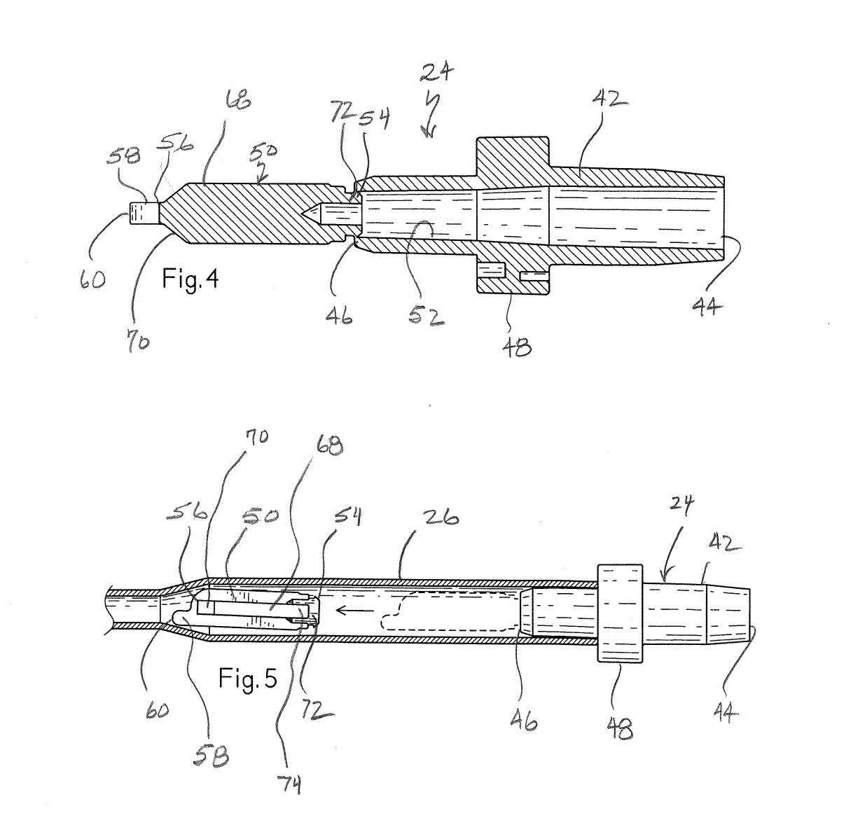

At the closed end 46 is found an elongate, rigid frangible member 50 closing the end from fluid flow by blocking an axial passageway or conduit 52 in the body 42 (FIG. 4). Included on the frangible member 50 is a closure end 54 preferably integrally joined to the closed end 46 of the body portion 42. The closure end 54 of the frangible member 50 is constructed, as by providing thinner walls or scored walls at the junction, to permit ready separation from the tubular body portion by manual manipulation of the frangible member relative to the body portion 42 when the frangible member is disposed inside the tube 26, for achieving user-activated opening of the connector 24.

Opposite the closure end 54 is a free end 56. The frangible member 50 has a longitudinal axis, extends in an axial direction “A” (FIG. 2) and has a member transverse dimension “T.”

A feature of the free end 56 is a finger portion 58 extending in the axial direction and having a transverse dimension “t” less than the member transverse dimension “T.” As seen in FIGS. 2 and 3, the finger portion 58 has a rounded or radiused tip 60 configured for easy passage within the flexible tube 26 once it is separated from the body portion 42. Also, the tip 60 is dimensioned to provide an axial standoff for the frangible member 50 once it reaches the next component in the administration set 10, such as the connector 22a, or a construction of the tube 26 caused by a line camp 30. This standoff feature facilitates flow within the tube 26 and around the separated frangible member 50 (best seen in FIG. 5).

Another feature of the finger portion 58 is that it is preferably located on the frangible member 50 to be transversely offset from the longitudinal axis of the frangible member. In other words, as seen in FIGS. 2 and 3, the finger portion 58 has a top surface 62 that is coextensive with an upper edge 64 of the frangible member 50.

Referring again to FIGS. 2 and 3, the frangible member 50 has opposing sidewalls 66, which are generally planar and parallel to each other, however other configurations are contemplated. At least one of the sidewalls 66 has a generally axially extending, laterally projecting standoff formation 68. In the preferred embodiment, the standoff formations 68 project perpendicularly from the sidewall 66. A main purpose of the standoff formations 68 is to position the separated frangible member 50 within the tube 26 as it flows to the connector 22a, and to promote fluid flow between the standoff formations and the corresponding sidewall 66. In addition, the standoff formations have tapered ends 70 pointing towards the free end 56 of the frangible member 50. For further facilitating medication flow within the tube 26 after the frangible member 50 is separated from the tubular body portion 42, the free end 56 of the frangible member is curved.

Opposite the finger portion 58, the frangible member closure end 54 defines a plug 72, and the standoff formations 68 each have a plug end 74 extending radially from the plug. Thus, the plug 72 is partially surrounded by the plug ends 74 and by extensions 76 of edge surfaces 64, 78 of the frangible member 50.

While a particular embodiment of the present frangible connector closure for a tubular connector has been shown and described, it will be appreciated by those skilled in the art that changes and modifications may be made thereto without departing from the invention in its broader aspects and as set forth in the following claims.

What is claimed:

1. A frangible connector closure, connected to a medication administration tube, comprising:
a tubular body portion having an open end configured for being connected to a supply of medication, and an opposite closed end;
an elongate, rigid frangible member disposed at said closed end and closing said end from fluid flow, said frangible member having a closure end and an opposite free end, said frangible member having a longitudinal axis, extending in an axial direction and having a member transverse dimension;
said free end forming a finger portion extending in said axial direction and having a transverse dimension less than said member transverse dimension; and
said closure end of said frangible member being constructed to permit ready separation from said body portion by manual manipulation of said frangible member relative to said body portion when said frangible member is disposed inside the tube, for user-activated opening of said connector.

2. The closure of claim 1 wherein said frangible member has opposing sidewalls, and at least one of said sidewalls has a generally axially extending, laterally projecting standoff formation.

3. The closure of claim 2 wherein said sidewalls are planar and parallel to each other, and said standoff formations project perpendicularly from said sidewalls.

4. The closure of claim 3 wherein said standoff formations have tapered ends pointing towards said free end.

5. The closure of claim 1 wherein said finger portion has a rounded tip.

6. The closure of claim 1 wherein said finger portion is transversely offset from said longitudinal axis of said frangible member.

7. The closure of claim 1 wherein said finger portion has a top surface coextensive with an upper edge of said frangible member.

8. The closure of claim 1 wherein said free end of said frangible member is curved.

9. The closure of claim 4 wherein said frangible member closure end defines a plug, and said standoff formations each have a plug end extending radially from said plug.

10. The closure of claim 9 wherein said plug is partially surrounded by said plug ends and by extensions of edge surfaces of said frangible member.
